(19) United States
(12) Patent Application Publication
(10) Pub. No.: US 2010/0313284 A1
(43) Pub. Date: Dec. 9, 2010

(54) MEANS AND METHODS FOR ELICITING AN IMMUNE RESPONSE

(75) Inventor: Menno Frederik Kielman, Voorschoten (NL)

Correspondence Address: RISSMAN HENDRICKS & OLIVERIO, LLP, 100 Cambridge Street, Suite 2101, BOSTON, MA, 02114, US

(73) Assignee: DeltaCell B.V., Velsen-Noord (NL)

(21) Appl. No.: 12/740,918

(22) PCT Filed: Oct. 31, 2008
(86) PCT No.: PCT/NL08/50682

§ 371 Date: Aug. 25, 2010

(30) Foreign Application Priority Data
Nov. 1, 2007 (EP) 07119816.2

(51) Int. Cl. A01K 67/00; A61K 39/00; A61K 39/395; C07K 16/00; C12N 5/0781; C12N 5/0783; A61K 35/12; A61P 37/04

(52) U.S. Cl. 800/13; 424/184.1; 424/277.1; 424/130.1; 530/387.1; 435/325; 424/93.7

(57) ABSTRACT

The invention provides means and methods for immunizing an animal against an antigen of interest, using stem cells, progenitor cells and/or dedifferentiated cells.

The invention relates to the fields of biology and immunology.

Higher vertebrates such as birds, rodents, primates and humans have developed a specialized immune system against antigens and pathogens that is known as the acquired immune system. The acquired immune system differs from the innate immune system present also in less developed species in that it increases in specificity and strength during repeated exposure to a given antigen. Once the defence is formed it persists and provides a lifelong immunity. The basis of the acquired immune system is formed by the production of antigen specific molecules produced by specialized cells, the T-lymphocytes and B-lymphocytes. Each T-lymphocyte, or T-cell, generates a different membrane bound T-cell receptor that is specific for a single antigenic epitope. Likewise, each B-lymphocyte, or B-cell, produces a different antigen specific antibody that initially is membrane bound but later during infection is secreted. Together with specialized antigen presenting cells, such as macrophages and dendritic cells, the T- and B-cells form an effective defence system against non-self antigens and pathogens.

This principle forms the molecular basis of vaccination where pre-exposure of pathogen-specific antigens induces a protective immune response before an actual infection with a pathogen occurs. The same principle is also used for the generation of polyclonal or monoclonal antibodies employing laboratory animals such as goat, rabbit or mouse, and the isolation of such antibodies. Polyclonal antibodies are synthesized by many different B-cells and can be isolated from serum. Monoclonal antibodies can be synthesized by a hybridoma cell line which is generated when an individual antibody producing B-cell is fused with a non-producing immortal B-cell.

The technique to produce antigen-specific poly- or monoclonal antibodies is invaluable for diagnostic and medical applications. To obtain a specific response against the native conformation of an antigen it is important to maintain its structural integrity and three-dimensional architecture. In addition, it is required that the antigen is, at least partly, purified to trigger an immune response that is strong and specific enough. For many proteins this is technically possible, although it may cost a considerable amount of time and money. However, for some proteins it is impossible to isolate them in a pure form due to their intrinsic properties. For instance, the structural integrity of many membrane proteins is dependent on their integration in the membrane. Isolation of these proteins often leads to misfolding of the protein and thus to a strongly reduced immune response against the native protein. Other proteins are embedded in a complex with other proteins and loose their antigenic epitope when the complex is disturbed.

One way to overcome the problems that occur by isolation and purification of antigens is by DNA-immunization. With this technique DNA encoding an antigenic epitope is transiently transfected to somatic cells of the body in order to direct expression of the antigenic epitope and to trigger an immune response. Although valuable, this technique has limitations with respect to the expression level and duration of the expression of the antigen. Therefore, this method is often not successful in triggering a desired immune response which is sufficiently strong, specific and long-lasting. Furthermore, normal somatic cells of the body need to be transfected which may cause cancer or other side effects in these cells. When somatic cells are cultured and transfected in vitro they stop dividing after about 40 cell divisions. Therefore it is not feasible to transfect normal somatic cells in vitro in order to obtain a high antigen expressing cell line for immunization. Existing cell lines, derived from a tumor or transfected with an oncogene, or obtained by inactivation of a tumor suppressor gene have been employed as a vehicle for immunization of an antigen. However, these cells are strongly immunogenic by themselves, mask the immunogenic reaction of the antigen to be expressed and therefore are not very efficient in triggering a specific immune response.

It is an object of the present invention to provide alternative means and methods for immunizing an animal against an antigen of interest.

To overcome the drawbacks of DNA immunization, and other cellular immunization protocols, the present invention provides means and methods with which it is possible to induce a specific immune response against as many polypeptides as desirable in their native conformation against low costs and with high efficiency. Accordingly, the invention provides a method for inducing an immune response—or enhancing an existing immune response—in an animal against an antigen of interest, comprising exposing said animal to a syngenic stem cell, a syngenic progenitor cell and/or a syngenic dedifferentiated cell, which stem cell, progenitor cell and/or dedifferentiated cell comprises a nucleic acid sequence encoding said antigen of interest, wherein said antigen of interest is exogenous to said animal and/or wherein said antigen of interest is not encoded by the germline of said animal.

In this invention we show that exposing an animal to a syngenic stem cell, progenitor cell and/or dedifferentiated cell comprising a nucleic acid sequence encoding an exogenous antigen of interest, or an antigen of interest which is not encoded by the germline of said animal, results in an immune response specific for said antigen.

To minimize the chance of inducing an immune reaction against the stem cells, the progenitor cells and/or the dedifferentiated cells, the cells employed for immunization are syngenic to the animal to be immunized. After administration, the stem cells, the progenitor cells and/or the dedifferentiated cells are preferably, but not necessarily, capable of proliferating and increasing in number in the animal. The stem cells, the progenitor cells and/or the dedifferentiated cells are preferably transfected with a nucleic acid construct which, in a preferred embodiment, comprises a promoter capable of driving the expression of a nucleic acid sequence encoding an antigen of interest. Most preferably, a strong constitutive promoter is used in order to obtain sufficient and long lasting antigen expression. The stem cells, the progenitor cells and/or the dedifferentiated cells are in vitro provided with a nucleic acid sequence encoding an antigen of interest. Before immunization, the stem cells, progenitor cells and/or dedifferentiated cells are preferably proliferated in vitro in order to select for antigen expressing clones with a desirable expression behavior with respect to, for instance, expression level, tissue specificity, developmental control, inducibility or repressibility. For the actual immunization, at least one clone is selected among the stably transfected clones with preferably a high expression level of an antigen of interest even after differentiation into numerous cell types and, preferably, subsequently expanded in vitro to obtain sufficient cells for the immunization procedure. Immunization is initiated by the administration of the stem cells to the animal. Preferably, these cells are injected subcutaneously, but other routes, such as intravenous injection or installation, are also applicable.

When the stem cells are administered, they preferably form locally a benign tumor, comprising differentiated and undifferentiated stem cells. The stem cells of this benign tumor do not spread through the body such as malignant tumor cells can do. Even the intravenous injection of a large number of cells does not result in malignant tumor formation throughout the body, indicating that the application of live stem cells to the animal does not result in disease. Other, adverse side effects in these cells are not observed either. Although the ES cells are syngenic in nature, they were suspected to trigger an immune response because of their abnormal growth and behaviour. This is illustrated by the many monoclonal antibodies have been generated in mice which recognize mouse epitopes on mouse antigens indicating that the acquired immune system can form antibodies against self-antigens. Contrary to this expectation the stem cells elicit very little to none immune response against themselves, even after differentiation into several cell types and growth in the animal for weeks. However they do elicit a strong immune response against both secreted and intracellularly expressed non-self antigens. The fact that even intracellular antigens elicit a specific immune response underscores the selectivity of the immune response induced by this method. This unexpected combination of 1) minimal recognition of self encoded antigens and 2) efficient recognition of both secreted and intracellular expressed non-self antigens makes this invention a breakthrough for the immunization of at least one, or multiple, antigens without the need for purifying the antigens. Furthermore, even though the stem cells, progenitor cells and/or dedifferentiated cells are transfected, they do not develop into malignant cells resulting in disease.

The present invention is suitable for generating an immune response against any antigen which can at least partly be encoded by a nucleic acid sequence. In one embodiment, syngenic stem cells, progenitor cells and/or dedifferentiated cells are stably transfected with a nucleic acid construct encoding at least one non-self antigen. Said antigen may be constitutively expressed or its expression may be rendered inducible by means of any regulatory sequence known in the art, such as for instance an exogenous inducer or repressor, so that the extent of antigen expression is regulated at will. Alternatively, the nucleic acid encoding the antigen may be integrated in the genome of said stem cell, progenitor cell or dedifferentiated cell, by homologous recombination, or directed integration using small recombinogenic sequences and proteins such as the Cre/Lox system, or by random integration, after which an endogenous promoter drives the expression of the antigen encoding nucleic acid. For homologous recombination the ROSA26 locus may be used to drive high level constitutive expression of the antigen in a predictable manner. However, a person skilled in the art may use other genomic loci as sites for homologous recombination. In other cases, for instance when the antigen is toxic for the cell, the promoter directing the expression of the antigen may either be tissue specific, developmentally controlled, inducible or repressible.

Non-limiting examples of nucleic acid sequences that are suitable for use to direct tissue specific expression are 1) The Surfactant Protein C promoter, which confers lung specific epithelial type-II cell expression, 2) the beta-globin Locus Control Region, which confers erythroid specific expression, 3) the CD2 locus control region which confers T-lymphocyte specific expression. Non-limiting examples of nucleic acid sequences that confer inducible gene expression are 1) the tetracyclin response elements in combination with the transactivator and/or suppressor, 2) the ecdyson response elements in combination with the transactivator, 3) the methallomethionine promoters which are activated by heavy metals. Another inducible system is for instance the Cre/Lox system which is suitable for excising or inserting a nucleic acid sequence essential for expression of a gene of interest.

The highest producing clone is preferably selected from the stably transfected cells in order to ensure strong stimulation of the immune system. However, there may be no need for selecting a clone with a desirable expression profile when the antigen encoding gene is transfected with high efficiency, for instance when a virus is used to transfect the stem cells. The only difference between the stem cells/progenitor cells/dedifferentiated cells and the host is the highly expressed antigenic epitope. Because of this marked difference, high expression and intimate interaction with the host's vascular and immune system, a strong immune response against said antigen is triggered.

This invention overcomes the need for purification of protein antigens and the costs involved in this. In addition this invention is invaluable for the generation of an immune response against epitopes having a conformation which depends on the natural environment of a cell, such as for instance epitopes of transmembrane proteins. The invention is also particularly suitable for generating an immune response against complex multipeptide or multiprotein epitopes, against peptide epitopes involving co-factors such as the chromophore of Green Fluorescent Protein, or against epitopes which are generated by the host cell as post-translational protein modifications, such as for instance glycosylation, palmitolyation, phosphorylation, ubiquitinalation, acetylation and others. Before the present invention, eliciting an immune response against such complexes was often cumbersome or impossible.

As used herein, an immune response against an antigen of interest is defined herein as an immunogenic reaction which is specifically directed against said antigen of interest and/or against a compound comprising an antigen of interest. For instance, a nucleic acid encoding an epitope is used for eliciting an immune response against a protein of interest. Said epitope need not to have exactly the same sequence as the corresponding region within said protein of interest. Immunogenicity of an epitope is for instance enhanced by altering at least one amino acid residue of said epitope. This is done with selection and screening methods which are well known in the art such as for instance a replacement technique. Said immune response preferably comprises the production of antigen-specific antibodies, B-lymphocytes and/or T-lymphocytes.

A syngenic cell of an animal is defined herein as a cell which originates from the same species and/or strain as said animal, or which originates from a related species and/or strain, whereby injection of said cell into said animal does not result in rejection of the cell by said injected animal. In one embodiment, said syngenic cell is an endogenous cell, meaning that said cell has been obtained from the same animal.

An antigen is exogenous to an animal if said antigen is not present within said animal when said animal is in a natural, healthy state, or if said antigen is normally not present or expressed in a certain part of said animal, or if said antigen is in a conformation which is not naturally present in said animal, or if said antigen comprises a secondary modification which is not naturally present in said animal. For instance, if an antigen is normally only expressed in liver cells, it is exogenous for other cell types of said animal. Hence, an antigen is exogenous when said antigen is expressed in a part of an animal where it is normally not expressed and/or present when said animal is in a natural, healthy state. An antigen is also exogenous when the conformation of the antigen is different than in its natural occurrence. For instance, an antigen not complexed with its natural partners is exogenous. An antigen is also exogenous when it has different modifications than in its natural occurrence, for instance if it has non-natural glycosylations, palmitolyations, acetylations and/or phosphorylations, or when it lacks some of the secondary modifications naturally present on the endogenous counterpart. An antigen not encoded by the germline of an animal for instance comprises an antigen which naturally occurs in cells of said animal, but which antigen has been altered in tumor cells within said animal. As used herein, the term “exogenous antigen” encompasses antigens which are not encoded by the germ line of the immunized animal.

Stem cells, progenitor cells and dedifferentiated cells are capable of differentiating into a more specialized cell. They are also capable of proliferating in vivo. The term “stem cell, progenitor cell and/or dedifferentiated cell” encompasses stem cells, progenitor cells, dedifferentiated somatic cells, undifferentiated cells, embryonic stem cells, adult stem cells, and tissue-specific stem cells. Said cell preferably comprises telomerase activity, either naturally or artificially acquired. In general, stem cells possess a natural telomerase activity which at least in part prevents telomere shortening and allows a stem cell to continue dividing.

There are no common genetic markers for all stem cells. Embryonic Stem (ES) cells and Primordial Germ (PG) cells have a high expression of Oct-4/Pou5f and nanog, however other stem cells such as lung type-II cells, or haematopoietic progenitor cells do not possess this activity. The distinction between stem cells and progenitor cells is based upon their flexibility in differentiation profile, i.e. the plasticity of the stem cell and not upon their ability to differentiate. Both stem cells and progenitor cells are capable of differentiating into another cell type, but stem cells are considered capable of differentiating into more different kinds of cell types as compared to progenitor cells.

In a preferred embodiment stem cells and/or progenitor cells comprising a nucleic acid sequence encoding an antigen of interest are injected subcutaneously into a syngenic host in order to form a teratoma. A teratoma is a benign tumor which is recognized as self and therefore not rejected. A teratoma does not cause a tumor-related disease either. In this embodiment the syngenic stem cells and/or progenitor cells act as an expression system for a nucleic acid construct encoding at least one exogenous antigen. The presence of said teratoma results in a strong, long-lasting immune response against said antigen of interest. Since syngenic stem cells and/or progenitor cells are used, no significant immune response is, in principle, elicited against the teratoma. Further provided is therefore a method for inducing an immune response—or enhancing an existing immune response—in an animal against an exogenous antigen of interest, comprising exposing said animal to a syngenic stem cell or progenitor cell, which cell comprises a nucleic acid sequence encoding said antigen of interest or a functional part or derivative thereof, wherein said stem cell and/or said progenitor cell is capable of forming a teratoma in said animal. Said teratoma is a benign tumor comprising cells derived from the applied stem cells and/or progenitor cells. Said teratoma may attract blood vessels and other cell types from the host. The size and growth of the teratoma formed depends on the number of stem cells and/or progenitor cells applied, the proliferative capacity of the stem cells and/or progenitor cells, the differentiation capacity of the stem cells and/or progenitor cells, and of the exogenous antigen expressed by the stem cells and/or progenitor cells.

Said teratoma is preferably formed by embryonic stem cells and/or primordial germ cells, since these cells are in general capable of faster proliferation as compared to other types of stem cells and progenitor cells. Embryonic stem cells and primordial germ cells are characterized by a high expression of Oct-4/Pou5f and nanog.

If stem cells, progenitor cells and/or dedifferentiated cells are used in order to vaccinate an individual against an antigen of interest, a growing teratoma is preferably not formed in view of safety and ethical reasons. Growth of the stem cells, progenitor cells and/or dedifferentiated cells is often undesirable. One of the reasons may be that the induction of a teratoma has to be avoided in case that a human being is immunized. The presence of a tumor may cause psychological problems. In other cases the presence of a teratoma in an animal is preferably avoided, for instance when the animal in question is important for the food industry. In such cases, non-proliferating stem cells, progenitor cells and/or dedifferentiated cells are preferably used. Therefore, in one preferred embodiment, syngenic stem cells, syngenic progenitor cells and/or syngenic dedifferentiated cells are used which are mitotically inactivated, so that they cannot proliferate. Many methods are available in the art for mitotically inactivating and/or killing cells. For instance, Mitomycine-C inactivation or X-ray (gamma) inactivation is used. In one embodiment, killed stem cells, killed progenitor cells and/or killed dedifferentiated cells are used. However, care should be taken during a killing process so that, after immunization, an animal will not be exposed too much to cell-derived antigens because that would interfere with the induction of an immune response against an antigen of interest.

Mitotically inactivated cells and/or killed cells according to the invention will remain inside an animal's body during a limited period of time, usually ranging from one day to several weeks, before they are broken down and removed by the animal's immune system. This is sufficient for inducing and/or enhancing an immune response against an antigen of interest.

Different kinds of stem cells and progenitor cells are suitable for performing a method according to the present invention. In a preferred embodiment, a syngenic stem cell, progenitor cell and/or dedifferentiated cell is used which is selected from the group consisting of embryonic stem cells, primordial germ cells, multipotent adult progenitor cells (MAPCs), embryonic progenitor cells, mesenchymal stem cells, mesenchymal progenitor cells, lung stem cells, lung progenitor cells, haematopoietic stem cells, hematopoietic progenitor cells, dedifferentiated somatic cells and immortalized somatic cells. Dedifferentiated somatic cells are somatic cells which have obtained the ability to differentiate into a more specialized cell. Such cell is for instance obtained by forced expression of at least one stem cell-related gene, such as for instance Oct-4, nanog and/or telomerase (TERT).

According to the present invention, the above mentioned cells are particularly suitable for eliciting a strong and long-lasting immune response which is specifically directed against at least one exogenous antigen of interest or which is specifically directed against an antigen which is not encoded by the germ line of an animal. Further provided is therefore a method according to the invention, wherein said cell comprises an embryonic stem cell, a primordial germ cell, a multipotent adult progenitor cell (MAPC), an embryonic progenitor cell, a mesenchymal stem cell, a mesenchymal progenitor cell, a haematopoietic stem cell, a hematopoietic progenitor cell, a dedifferentiated somatic cell and/or an immortalized somatic cell. Most preferably, a syngenic embryonic stem cell and/or a primordial germ cell is used. Embryonic stem cells and primordial germ cells comprising a nucleic acid sequence encoding at least part of an exogenous antigen of interest are capable of forming a teratoma upon subcutaneous injection into a syngenic host. As a result, a strong, specific immune response against said exogenous antigen of interest is elicited.

A method according to the invention is suitable for generating a broad and strong immune response against a pathogen. In this embodiment stem cells, progenitor cells and/or dedifferentiated cells are used comprising a nucleic acid sequence encoding at least one antigen derived from a pathogenic organism. Said antigen does not need to have a sequence which is identical to the corresponding part of a pathogenic protein, as long as said antigen is capable of inducing and/or enhancing an immune response that is specific for said pathogen. Preferably, multiple independent pathogen-specific antigens are expressed in the same cell. When an animal is exposed to such cell, a broad pathogen-specific immune response is elicited. Alternatively, or additionally, an animal is provided simultaneously with at least two different antigen-expressing stem cells, progenitor cells and/or dedifferentiated cells. Hence, it is possible to use cells which comprise nucleic acid sequences encoding different antigens derived from the same pathogen. Administration of at least two cells, wherein each cell expresses a different pathogen-specific antigen, results in a pathogen-specific immune response which is broader as compared to the situation where only one pathogen-specific antigen is expressed by syngenic stem cells, progenitor cells and/or dedifferentiated cells.

In another embodiment it is desirable to express a fusion protein as the exogenous antigen. A part of this fusion protein is preferably strongly immunogenic, and/or favours the exposure of the antigen to the immune system. The immune response against the other part of the fusion protein may therefore be enhanced. Examples of proteins which are strongly immunogenic are KLH and ovalbumine. A fusion protein may also be formed with a traceable protein, for instance a reporter protein or a protein with an enzymatic activity. An advantage of using these traceable fusion proteins is that the expression and activity of the fusion protein as a whole can be followed in vitro and in vivo. In this way it is possible to follow the immune response against the fusion protein since this will inactivate the activity of the fusion protein as a whole. Traceable proteins that may be used as part of such a fusion protein are for instance; luciferases, e.g. Renilla luciferase and firefly luciferase; Beta-galactosidase; alkaline phosphatase; peroxidase and Chloramphenicol Acetyl Transferase (CAT)

As stated before, for some proteins it is impossible to isolate them in a pure form due to their intrinsic properties. For instance, the structural integrity of many membrane proteins is dependent on their integration in the membrane. Isolation of these proteins often leads to misfolding of the protein and thus to a non-specific immune response. Other proteins are embedded in a complex with other proteins and loose their antigenic epitope when the complex is disturbed. A method according to the invention is particularly suitable for eliciting and/or enhancing an immune response against such proteins, because they are expressed in a cellular environment. Membrane-bound proteins, for instance, are incorporated into the membrane of the cells. As a result, their native epitopes are exposed and an efficient immune response is elicited. One embodiment therefore provides a method according to the invention, wherein said antigen of interest cannot be purified without disturbing its structure. In one preferred embodiment said antigen of interest is an epitope of a membrane-bound protein.

A method according to the invention is also suitable for generating an immune response against an antigen expressed by a malignant cell. One preferred embodiment therefore provides a method according to the invention, wherein said antigen of interest comprises an antigen expressed by a malignant cell.

An animal suitable for a method according to the invention comprises any animal capable of eliciting an immune response against an exogenous antigen. Preferably, said animal is capable of producing B-lymphocytes, T-lymphocytes and/or antibodies. When the method according to the invention is used for producing B-lymphocytes, T-lymphocytes and/or antibodies of interest, preferably a non-human animal is used. Such non-human animal preferably comprises a mammal or a bird, with commercial or emotional value. Examples of animals with commercial or emotional value known are laboatory animals, animals used for in agriculture, poultry farming, vetirinary medicin and animals kept as pet or used in sports. This includes domestic animals, semi-domestic animals, captive wild animals and animals living in the wild, such as whales and seals. Examples of important laboratory animals are mice, rats, guinea pigs, rabits, goat and llama. Examples of animals important for agriculture are hoofed animals such as bovine, horse, pig, reindeer and sheep. Important animals for poultry farming are chicken, turkey, goose and ducks. Important animals employed as pet or in sports are dogs, cats, and horses.

In one embodiment, however, a method according to the invention is used for immunizing a human individual. In this embodiment, said human individual is vaccinated in order to elicit a protective immune response, for instance against a disease-associated antigen. In another embodiment, an animal with a commercial or emotional value, such as for instance cattle or a pet, is vaccinated in order to elicit a protective immune response, for instance against a disease-associated antigen. For vaccination purposes, mitotically inactivated and/or killed cells are preferably used, so that the cells will barely—if at all—proliferate and will be cleared from the body.

Once an immune response has been induced in a non-human animal using a method according to the invention, antibodies, B-lymphocytes and/or T-lymphocytes which are specific for an antigen of interest are preferably obtained for further use. Methods and protocols for harvesting antibodies, T-cells and/or B-cells, isolation and purification protocols are well known in the art. For instance, B-cells are isolated from a sample by selection for CD19 (B-cell marker) and/or cell surface IgG and/or CD27 (to mark memory cells). Pure isolates of B-cells and T-cells can also be obtained by negative selection against all other cells present in the crude cell sample, for instance using cell surface protein markers not or lowly expressed by the B-cells or T-cells. Furthermore, an antibody, B-cell and/or T-cell capable of specifically binding an antigen of interest is for instance selected in a binding assay using said antigen of interest. This is done using any method known in the art, for instance an ELISA. In one embodiment antibodies, B-cells and/or T-cells are incubated with a labelled antigen. Bound antibodies, B-cells and/or T-cells are subsequently detected and/or isolated via said label. In one embodiment IgM producing B-cells and/or IgG producing B-cells are selected and/or isolated. Preferably an IgG producing B-cell is selected and/or isolated, for instance using surface markers, using methods known in the art.

In a preferred embodiment, antibodies, T-cells and/or B-cells which are specific for an antigen of interest are isolated and further used for human benefit. For instance, the genes encoding the Ig heavy and/or light chains are isolated from a harvested B-cell and expressed in a second cell, such as for instance cells of a Chinese hamster ovary (CHO) cell line. Said second cell, also called herein a producer cell, is preferably adapted to commercial antibody production. Proliferation of said producer cell results in a producer cell line capable of producing antibodies of interest. In a particularly preferred embodiment, monoclonal antibodies are produced. Preferably, said producer cell line is suitable for producing compounds for use in humans. Hence, said producer cell line is preferably free of pathogenic agents such as pathogenic micro-organisms.

Alternatively, or additionally, nucleic acid encoding the T-cell receptor is isolated from a harvested T cell of interest and incorporated into naive (preferably human) T-cells. The T-cells are preferably cultured in order to obtain a T-cell line.

Further provided is therefore a method according to the invention, further comprising obtaining from said animal an antibody, a polyclonal antibody, a B-lymphocyte, and/or a T-lymphocyte specifically directed against said antigen of interest. In one embodiment said obtained B-lymphocyte and/or T-lymphocyte is immortalized, for instance via Epstein-Barr virus (EBV) transformation. In a particularly preferred embodiment said B-lymphocyte is used to generate monoclonal antibodies. This is for instance done using hybridoma technology.

Once a B-lymphocyte capable of producing antibodies that are specifically directed against an antigen of interest is obtained, it is preferably used for the production of antigen of interest specific antibodies. As explained above, the genes encoding the Ig heavy and/or light chains of said B-lymphocyte are preferably isolated and expressed in a second cell, called herein a producer cell, which is adapted to commercial antibody production. Further provided is therefore a method according to the invention, further comprising obtaining antibodies produced by said B-lymphocyte or produced by a cell comprising a gene encoding an Ig heavy chain and/or an Ig light chain of said B-lymphocyte.

B-lymphocytes, T-lymphocytes and/or antibodies which are obtained by a method according to the invention, and/or functional parts or derivatives thereof, are preferably used for the preparation of a medicament and/or prophylactic agent. Said medicament and/or prophylactic agent for instance comprises B-lymphocytes, T-lymphocytes and/or antibodies which are specifically directed against an antigen which is associated with disease, such as for instance an antigen of a pathogenic organism or a tumor-associated antigen. A method according to the invention therefore preferably further comprises preparing a medicament or prophylactic agent comprising said B-lymphocyte, T-lymphocyte and/or said antibody, or a functional part or derivative thereof. Such antibody, B-lymphocyte, T-lymphocyte, functional part, derivative, medicament or prophylactic agent is preferably administered to an individual suffering from, or at risk of suffering from, a disorder associated with the presence of said antigen of interest.

An antibody, B-lymphocyte, T-lymphocyte, functional part, derivative, medicament or prophylactic agent obtained with a method according to the invention is also herewith provided. In one preferred embodiment said antibody, B-lymphocyte, T-lymphocyte, functional part, derivative, medicament or prophylactic agent is used for therapeutic and/or prophylactic applications. An antibody or a T-lymphocyte obtained by a method according to the invention, or a functional part or derivative thereof, for use as a medicament or prophylactic agent is also provided. Said antibody or T-lymphocyte specifically directed against an antigen of interest, obtained by a method according to the invention, or a functional part or derivative thereof, is preferably used for the preparation of a medicament or prophylactic agent for counteracting or at least in part preventing a disorder associated with the presence of said antigen of interest in an individual. Said individual preferably comprises a human individual.

Stem cells, progenitor cells and dedifferentiated cells according to the invention, comprising a nucleic acid sequence encoding an antigen of interest, are also particularly suitable for therapeutic and/or prophylactic applications, since they are capable of eliciting and/or enhancing an immune response specifically directed against said antigen of interest. By eliciting or enhancing such immune response, a disorder associated with the presence of said antigen of interest is counteracted or at least in part prevented. A stem cell or progenitor cell or dedifferentiated cell comprising a nucleic acid sequence encoding an antigen of interest, for use as a medicament or prophylactic agent, wherein said stem cell or progenitor cell or dedifferentiated cell is syngenic to the recipient and said antigen of interest is exogenous to the recipient and/or not encoded by the germ line of the recipient is therefore also provided. Said stem cell, progenitor cell or dedifferentiated cell comprising a nucleic acid sequence encoding an antigen of interest is preferably used for the preparation of a medicament or prophylactic agent for counteracting or at least in part preventing a disorder associated with the presence of said antigen of interest in an individual, wherein said stem cell or progenitor cell or dedifferentiated cell is syngenic to said individual and said antigen of interest is exogenous to said individual and/or not encoded by the germ line of said individual. Further provided is therefore a medicament or prophylactic agent comprising a stem cell or a progenitor cell or a dedifferentiated cell and a suitable carrier, diluent or excipient, wherein said stem cell or progenitor cell or dedifferentiated cell is syngenic to the recipient and wherein said stem cell or progenitor cell or dedifferentiated cell comprises a nucleic acid sequence encoding an antigen of interest which is exogenous to the recipient and/or not encoded by the germ line of the recipient.

As already described herein before, various different kinds of stem cells, progenitor cells and dedifferentiated cells are suitable for the above mentioned therapeutic and prophylactic applications. In a preferred embodiment, a syngenic stem cell, progenitor cell and/or dedifferentiated cell is used which is selected from the group consisting of embryonic stem cells, primordial germ cells, multipotent adult progenitor cells (MAPCs), embryonic progenitor cells, mesenchymal stem cells, mesenchymal progenitor cells, haematopoietic stem cells, lung stem cells, hematopoietic progenitor cells, dedifferentiated somatic cells and immortalized somatic cells. According to the present invention, these cells are particularly suitable for eliciting a strong and long-lasting immune response which is specifically directed against at least one antigen of interest. Most preferably, a syngenic embryonic stem cell and/or a syngenic primordial germ cell is used. Embryonic stem cells and primordial germ cells comprising a nucleic acid sequence encoding at least part of an (exogenous) antigen of interest are capable of forming a teratoma upon subcutaneous injection into a syngenic host. As a result, a strong, specific, neutralizing immune response against said antigen of interest is elicited.

Methods for counteracting and/or preventing a disorder associated with the presence of an antigen of interest using stem cells, progenitor cell, dedifferentiated cells, antibodies or T-lymphocytes according to the invention are also provided. One embodiment therefore provides a method for counteracting and/or preventing a disorder associated with the presence of an antigen of interest, comprising administering to an animal suffering from, or at risk of suffering from, said disorder a therapeutically effective amount of a syngenic stem cell or syngenic progenitor cell or syngenic dedifferentiated cell comprising a nucleic acid sequence encoding said antigen of interest, or a therapeutically effective amount of an antibody or T-lymphocyte, or a functional part or derivative thereof, specifically directed against said antigen of interest obtainable by a method according to the invention. A method for vaccinating an animal against an exogenous antigen of interest or multiple exogenous antigens of interest, comprising administering to said animal a syngenic stem cell or syngenic progenitor cell or syngenic dedifferentiated cell comprising at least one nucleic acid sequence encoding said antigen of interest or encoding multiple antigens of interest is also provided.

A further embodiment provides a non-human animal wherein an immune response is raised against an exogenous antigen of interest (for instance an antigen which is not encoded by the animal's germ line), said non-human animal comprising a stem cell or progenitor cell or dedifferentiated cell that is syngenic to said animal, said cell comprising a nucleic acid sequence encoding said exogenous antigen of interest. As already explained, said animal according to the invention is particularly suitable for producing antibodies, B-cells and/or T-cells against an antigen of interest, preferably against an antigen having a conformation which depends on the natural environment of a cell, such as for instance a transmembrane protein and/or a complex multipeptide or multiprotein epitope.

The invention is further explained in the following examples. These examples do not limit the scope of the invention, but merely serve to clarify the invention.

### BRIEF DESCRIPTION OF THE DRAWINGS

FIG. 1: Growth of teratoma's in mice 0, 1, 2, 3, 4, and 5.

FIG. 2: Effect of SC injection of H8L46 ES cells on Renilla luciferase activity in serum

Sera of mice drawn at the indicated time points were analyzed for their Renilla luciferase activity as described in Material and Methods. Data of the SC H8L46 injected mice were compared to data obtained with serum of 2 PBS injected mice. The levels of luminescence of the sera of the 2 control mice never exceeded 800 AU (data not shown).

FIG. 3: Presence of neutralizing antibodies in serum of SC H8L46 injected mice Sera obtained at time of sacrifice of the mice were examined for the presence of Renilla luciferase neutralizing antibodies as described in Material and Methods. Renilla sup incubated with PBS had a luminescence of 1100 AU. Three mice reduced this level (mouse 0,3 and 5) completely and mouse 1 also had some reducing activity.

FIG. 4: Detection of Renilla and GFP directed antibodies in the spleen cell culture medium of teratoma positive mice. Per lane 50 ng recombinant Renilla-GST and 25 ng recombinant GFP was loaded. Blots were probed with the spleen cell culture medium of the indicated mice or with commercially available antibodies against Renilla luciferase and GFP.

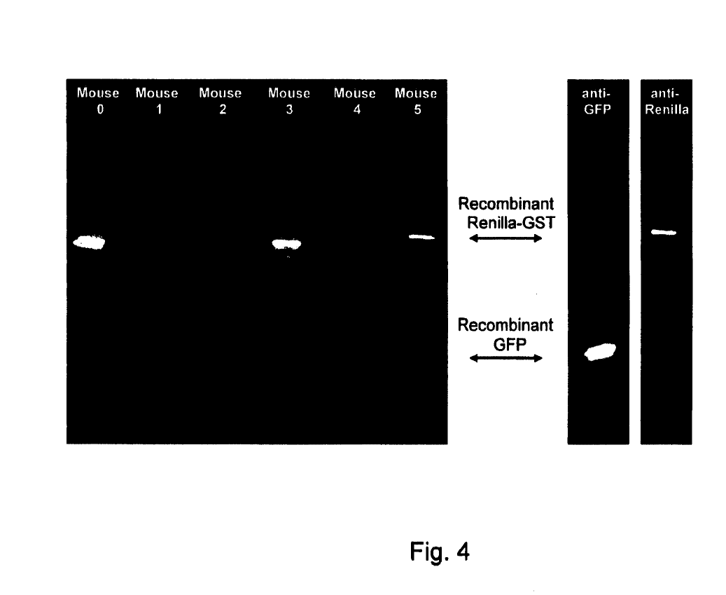

FIG. 5: Detection of GFP and Renilla luciferase neutralizing antibodies in the spleen cell culture medium of teratoma bearing mice. Whole cell lysates of H129/SVEV (1), H8L46 (2), 293T (3), 293T transfected with GFP (4), Renilla (5) or both (6) were subjected to Western blot analysis. Lane 7 contains a mixture of 50 ng recombinant Renilla-GST and 25 ng recombinant GFP. Marker lanes are indicated by M. Blots were probed with the spleen cell culture medium of the indicated mice.

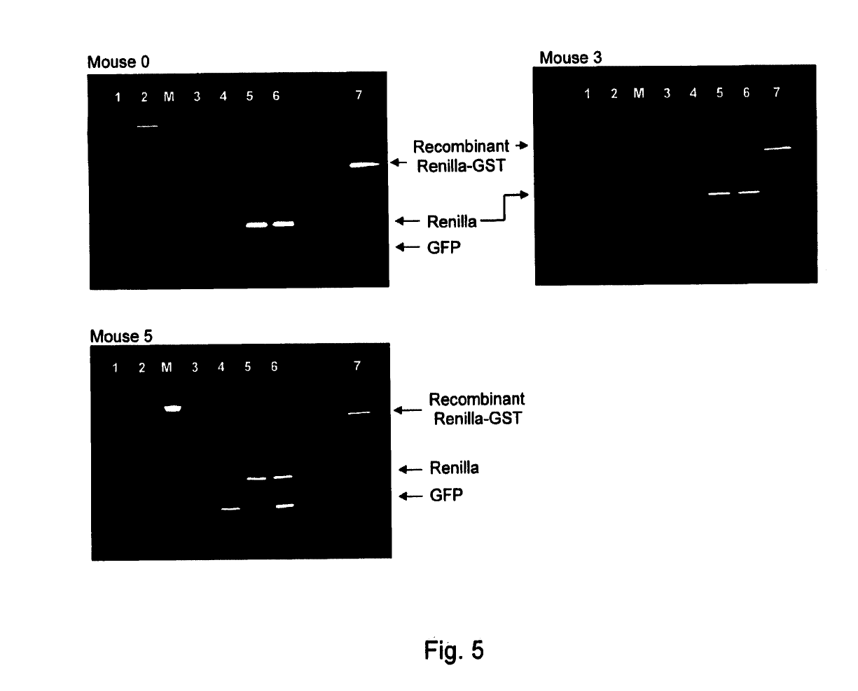

FIG. 6: validation of the Renilla and GFP Elisa's.

FIG. 7: Isotype determination of three monoclonal antibodies with affinity for GFP.

FIG. 8: Body weight of mice during the study. The body weight of the mice in control group (0) or the treatment group (o) was not influenced by the vaccination method. Arrows indicate the time of vaccinations.

FIG. 9: Tumor development in C57BL/6 mice. Mice were challenged with 1×10⁵ live B16F10 melanocytes. The growth of the tumours in vaccinated mice, shown as the weight of the tumours, were markedly reduced as compared to the growth of tumours in the control mice (p=0.05). Shown are the mean weight of the tumors in mg and the SEM.

### EXAMPLES

### Example 1

### Induction of Neutralizing Antibodies Against Secreted Renilla Luciferase Expressed by Syngenic Embryonic Stem Cells

### Introduction:

Immunization of mice is often performed by injection of a purified protein, the antigen. Depending on the protein a strong or weak immune reaction is triggered.

The strength of the immune reaction is for instance measured by the presence of antigen-specific antibodies in serum. For weak antigenic proteins an adjuvant is often co-injected which sensitizes the immune response and thereby increases the response to the actual antigen. Addition of an adjuvant, such as Freunds complete adjuvant is not always allowed by legal law and therefore not always applicable.

Here we have applied a new immunization technique in which we use embryonic stem cells as a vehicle to express an antigen. Embryonic stem cells are undifferentiated cells with a normal karyotype that have the ability to differentiate to all possible cell-types. When embryonic stem cells (ES cells) are injected into a mouse strain from which the ES cells originally have been isolated, i.e. an isogenic strain, a benign tumor is formed comprising differentiated and undifferentiated cells. The differentiation in such a teratoma occurs randomly and results in many different cell types and tissues. The main blood supply of the teratoma is provided by the ingrowth of bloodvessels from the host. When the teratoma becomes too large some areas in the teratoma may become necrotic because of insufficient oxygen levels. The necrotic or apoptotic cells are subsequently removed by the immune system of the host. Like tumor based cell lines it is possible to modify the genetic content of ES cells by transfection with recombinant DNA and clonal selection of the transformants using selectable marker genes.

In order to determine whether embryonic stem cells can be used as a vehicle to express exogenous proteins we have transfected mouse embryonic stem cells with DNA constructs expressing Green Fluorescent Protein (GFP) and secreted Renilla Luciferase. GFP is a protein originally isolated from the jellyfish Aequera Victoria that fluorescents when exposed to blue light. The gene encoding GFP is often used as a marker gene to identify cells or individual proteins. Here we have created GFP-expressing embryonic stem cells in order to identify these cells when injected in a non-fluorescent host. The GFP positive cells were subsequently transfected for a second time with the gene coding for secreted Renilla luciferase.

Renilla luciferase protein is a light emitting protein normally expressed by the sea pansy (Renilla reniformis) as a defence system against predators. The Renilla Luciferase utilizes coelanterazine as a substrate to emit light that can be sensitively measured and quantified. The double transfected GFP/sRenilla embryonic stem cells thus constitutively express two heterologous proteins which can be measured by their fluorescent and light emitting properties. Since the embryonic stem cells are isogenic for the Ola129/sveF mouse strain, GFP and sRenilla luciferase are the only proteins that are non-self for this mouse strain.

The renilla luciferase is secreted by the embryonic stem cells and thus is fully exposed to the immune system. The GFP is not secreted and thus more hidden for the immune system, although the GFP from necrotic/apoptotic cells will eventually be exposed. The sRenilla synthesized by the cells of the teratoma is partly secreted in the serum and its concentration can be measured with a luminometer from a tail blood sample. In this experiment we have followed the presence of antibody specific for Renilla and GFP in the serum of mice immunized with syngenic GFP/sRenilla expressing ES cells by measuring their neutralizing effect on sRenilla bioluminescence activity, by immune fluorescence microscopy and by western blotting.

### Material and Methods:

### Generation of Embryonic Stem Cells Expressing Constitutively GFP and Renilla Luciferase.

ES cells from the 120\SVEV mouse strain (specialty media) were transfected with constitutive expression constructs encoding sRenilla and GFP under control of the strong chimeric beta-actin/CMV (CAG) promoter. The selection of stably transfected ES cells was performed with Hygromycin for the GFP clones and Puromycin for the sRenilla clones. The stably transfected GFP-ES clones were screened for high GFP expression by immune fluorescence microscopy and confirmed to maintain expression of GFP after differentiation. The best GFP expressing clone was used to transfect the sRenilla construct and the highest sRenilla expressing clones were selected by testing the supernatants for sRenilla luminescence activity, resulting in a clone called, H8L46.

### Culture of the ES Cells Expressing Renilla Luciferase and GFP (H8L46) on Mitotically Inhibited STO Cells:

The following protocol was used:
Coat a 9 cm culture dish (Greiner) by adding 3.5 ml of 0.1% gelatin (Sigma) covering the whole plate, incubate at least 10 min
Remove the gelatin and add 5 ml of complete culture medium (containing per bottle of 500 ml of DMEM (Gibco): 5 ml glutamine, 5 ml Penicillin-Streptomycin, 5 ml Non-Essential Amino Acids, 5 ml Nucleosides (all supplied by Specialty Media), 5 ml LIF (Chemicon), 5 ml Beta 2-Mercaptoethanol and 50 ml FCSi (both supplied by Sigma))
Thaw the mitotically arrested STO cells (ATCC) in a 37° C. water bath and re-suspend in 15 ml complete medium, add 5 ml of cell suspension per plate (±1.6×10⁶ cells per plate)
Incubate at least 4 hours in a CO₂ incubator at 37° C.
Thaw the H8L46 (generated by transfection of the 129/SVEV embryonic stem (ES) cells (Specialty Media) with a plasmid coding for secretable Renilla Luciferase cells and a plasmid coding for GFP) in a 37° C. water bath re-suspend in 10 ml complete medium
Remove the medium of STO cell layered 9 cm culture dish
Add the ES cells to the ‘empty’ 9 cm dish and incubate in a CO₂ incubator at 37° C.
After 2 days replace the culture medium without washing the cells

### Collection of H8L46 and Preparation of Inoculum.

Collect H8L46 cells by trypsin/EDTA (Gibco) treatment and determine the cell numbers. Wash the cells 3 times with phospate buffered saline (PBS, Gibco) and prepare a cell suspension of 2.5×10⁶ H8L46 cells per 200 μl in PBS.

### Terato Induction.

Animals were housed under the guidelines of the Animal facility of the Leiden University Medical Centre (PDC-LUMC) according to the Dutch law.

The mice were F1 offspring of a Swiss (female)*Ola/129 (male) (Harlan) mating generated at the Animal facility of the LUMC. Mice were subcutaneously (SC) injected with 200 μl (i.e. 2.5×10⁶ cells) near the left or right hind leg. Animals were examined twice a week and ones every 2 weeks blood was drawn by tail incision and blood was collected in EDTA coated vials (Sarstedt). After 7 weeks the mice were euthanised, blood was drawn by a heart punction and spleens were isolated.

### Handling of Spleens and Spleen Cell Culture

After isolation, the spleens were washed in sterile PBS and minced utilizing a cell strainer of 100 μm mesh (Greiner). Cell suspensions were plated in a 9 cm culture dish in DMEM/F12 (Gibco) supplemented with 2% FCS, Penicillin-Streptomycin. After 3 days of culture, the medium was collected and cell debris was removed by filtering through a 0.45 um Filter (Nunc). This conditioned medium is referred to as the Spleen Cell Supernatant.

### Determining for the Presence of Neutralizing Antibodies Directed Against Renilla Luciferase in Spleen Cell Culture Supernatant (SCCS) and Mouse Serum

H8L46 culture supernatant is diluted 100 times in PBS. This diluted culture supernatant harbors Renilla luciferase activity as the H8L46 cell line secretes its produced Renilla luciferase into the culture medium. The diluted culture supernatant of H8L46 will be referred to as Renilla sup. Two and a half μl of mouse serum was incubated for 1 hour with 20 μl of Renilla sup to enable formation of antibody/Renilla luciferase complexes. Next 10 μl of the mixture is added to 40 μl of RLB and in this sample the activity of the Renilla luciferase is determined by adding 50 μl of substrate utilizing a luminescence plate reader (Berthold). To determine Renilla luciferase activity in the Renilla sup, 20 μl of Renilla sup was incubated for 1 hour with 2.5 μl PBS instead of serum sample. Ten μl of this mixture was used for analyzing luminescence. Since the mouse serum may contain Renilla luciferase activity, 20 μl of these samples were incubated for 1 hour with 2.5 μl PBS and 10 μl of this mixture than was used to analyze luminescence.

Serum samples were also directly analyzed for their content of Renilla luciferase activity. For this 10 μl of serum was incubated with 40 μl of RLB and than luminescence was determined as described.

Transfection of Stem Cells and Non Stem Cells

To determine whether SCCS contain antibodies directed against GFP and/or Renilla luciferase the parental ES line 129/SVEV and the non related cell line 293T were transfected with a plasmid coding for either 1 of the proteins. The Renilla luciferase construct used in the transfections codes for a non-secreted protein therefore cells have to be permeabilized prior to detection. To transfect the 129/SVEV cells lipofectamin (Invitrogen) and for the 293T PPei (Polysciences) was used as transfection agent.

Cells were grown in 12 well plates and per transfection 0.4 μg DNA was used. DNA as well as the transfection agents were diluted in medium without supplements. After diluting DNA and transfection agents, dilutions were incubated for about 5 min at room temperature. Next DNA and the transfection agent were mixed and incubated for 20 min at room temperature. The complexes of DNA/transfection agent were added to the cells and were incubated for 48 hours at 37° C. in a CO₂ incubator.

Determining for the Presence of Antibodies Directed Against GFP and/or Renilla Luciferase Utilizing Immune Fluorescence Microscopy

Target cells are seeded in wells of a 48-well plate (Costar?) and after overnight adherence fixed with 1% paraformaldehyde (Sigma) in PBS, for 10 minutes. In order to permeabilise the cells, the cells were washed with PBS supplemented with 0.05% Tween-20 (wash buffer).and subsequently incubated for 10 minutes with PBS supplemented with 0.01% Triton-X100. Thereafter cells were washed with wash buffer and incubated for 1 hour with PBS supplemented with 2% normal goat serum (Jackson) to block a-specific binding places.

The SCCS was either used undiluted or diluted in wash buffer with control regular culture medium either undiluted or diluted. The target cells are incubated with 250 μl of either culture medium or SCCS for 3 hours. After 3 consecutive washes with wash buffer cells are incubated for 1 hour with a 1/100 diluted Rhodamine-Red-X labelled goat-anti-mouse secondary antibody (Jackson).

The cells were washed twice and stored at −20° C. until examination utilizing a fluorescence microscope (Zeiss).

### Results:

Immunization of Mice with H8L46 Cells

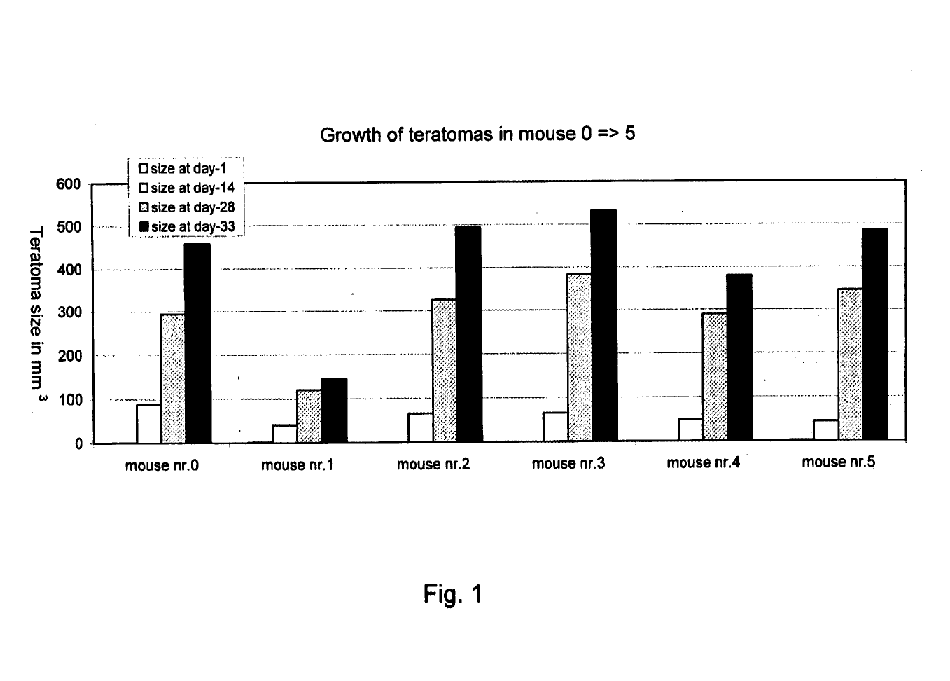

Six female mice of 6 months of age were injected with 2.3×10⁶ H8L46 ES cells (GFP⁺/sRenilla⁺). The growth of the teratomas was determined over time and all 6 mice developed palpable teratomas over a 6 weeks period (FIG. 1). The teratoma sizes of 5 mice were comparable and one mouse Nr-1 developed a smaller teratoma.

### Neutralization of Serum Renilla Activity During Teratoma Growth.

Mouse sera obtained at various stages during growth of the teratomas were analyzed for their specific Renilla luciferase activity secreted by the teratomas.

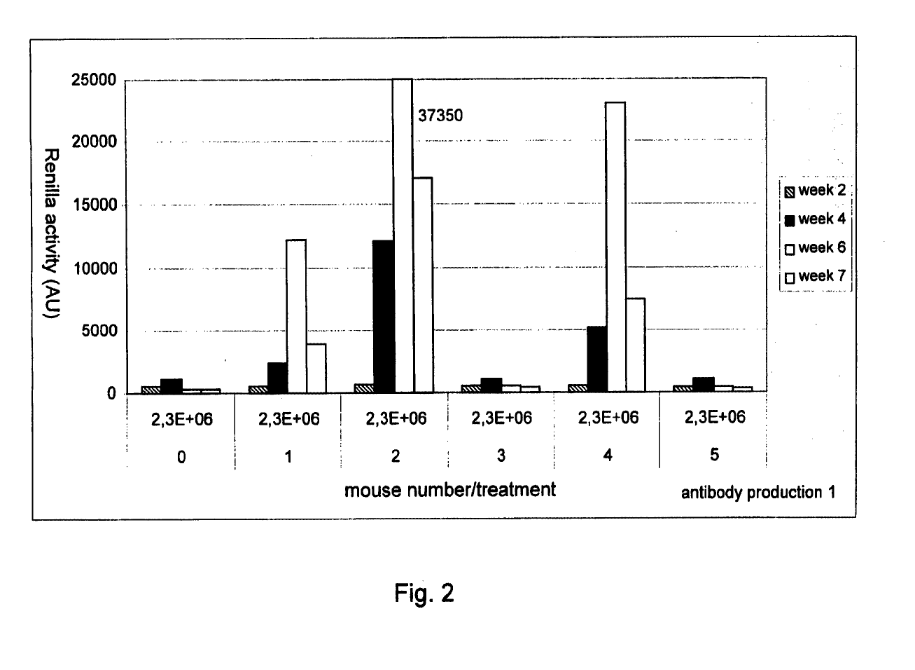

In three mice, mouse nr.-1, nr.-2 and nr.-4, high levels of luciferase activity were detected in comparison with mice nr.-0, nr.-3, and nr-5 (FIG. 2). There is no correlation between teratoma size and serum luciferase activity. For instance, mouse-1 developed a four times smaller teratoma than mouse-0, mouse-3 and mouse-5 but reached about 30 times higher serum luciferase values than mouse-0, mouse-3 and mouse-5. Remarkably, in all six mice the relative luciferase levels declined although the teratomas were still gaining in size, suggesting that the mice developed neutralizing antibodies against the sRenilla. The mice with relatively low luciferase levels showed a earlier decline than the mice with relatively high levels of sRenilla in the serum. After 7 weeks the mice were sacrificed and their serum was collected, together with the spleen. In this final serum the luciferase activity of mouse-0, mouse-3 and mouse-5 were comparable to the control mice indicating that most of the secreted Renila from the teratoma was neutralized.

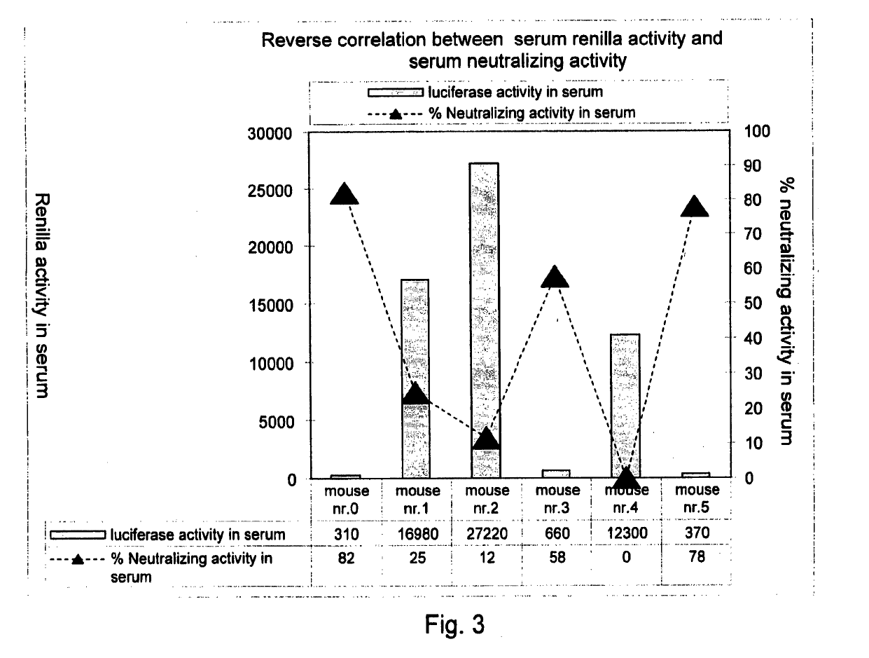

In order to analyze whether the final sera had neutralizing activity the sera were incubated with a culture supernatant of the H8L46 ES cells (GFP⁺/sRenilla⁺) containing sRenilla activity. The results showed that the mice with the lowest levels of sRenilla activity in the final sera, contained the highest sRenilla neutralizing activity. About 80% of the added sRenilla activity was inhibited with sera from mouse-0 and mouse-5. Only one mouse (nr-4) showed no inhibition of sRenilla activity (FIG. 3). Together these results show that the H8L46 ES-cells expressing GFP and sRenilla are not rejected in syngenic mice however they developed an immune response against at least secreted Renilla luciferase. This immune response in three out of six mice almost completely inhibited the sRenilla activity. In the other mice (nr.-1, nr.-2 and nr-4), the development of neutralizing activity was less clear. However, other antibodies may have been raised in these mice which recognize but do not inhibit sRenilla and cannot be measured with this test.

### Example 2

Analyzing Culture Supernatant of Spleen Cells Obtained from SC H8L46 Injected Mice

In order to detect whether other Renilla and GFP specific antibodies were induced, the spleen cells of these mice were collected and cultured in vitro to collect a conditioned medium containing the antibody repertoire synthesized by the B-cells at the moment of sacrifice. Spleens were collected minced and isolated cells cultured for 3 days where after supernatant was collected.

The spleen cell supernatants were incubated with various target cells expressing GFP and/or Renilla luciferase in order to detect specific anti-GFP and anti-Renilla antibodies. In addition we wanted to establish whether there was an antibody response raised against the ES 129/svev background cells that formed the expression vehicle of the GFP and sRenilla constructs.

The results are depicted in Table 1. The spleen cell supernatants of mice 0,1,3,4 and five showed no reactivity against the ES-129/svev background indicating that these cells were not immunorective. Only one mouse, mouse nr.-2, developed an immune response indicating that a-specific antibodies were present in this mouse serum. We could not establish whether these antibodies pre-existed before immunization because of the unavailability of pre-immune spleen cell supernatant. We conclude that with exception of spleen cell supernatant-2, all other supernatants contained antibodies specific for GFP and/or Renilla luciferase.

In order to detect to which protein the response was directed 129/SVEV cells were transiently transfected with either GFP or non-secreted Renilla luciferase expression constructs. The spleen cell supernatants of mouse 0, 1, 3 and 5 stained the GFP transfected cells moderately to strong. Spleen cell supernatant of mouse 4 was unable to detected GFP expression (Table 1). In all cases there was a correlation with the level of GFP expressed by the transfected cells and the strength of the immunostaining with the spleen cell supernatant (Table 1). Cells expressing Renilla luciferase were not detected by the spleen cell supernatants of mouse-1, -4 and -5 while the spleen cell supernatants of mouse-0 and mouse-3 stained the cells moderately. The spleen cell supernatant of mouse 2 stained the 129/svev transfected with either GFP or Renilla luciferase strong to very strong indicating the presence of a-specific antibodies in the spleen cell supernatant of this mouse.

TABLE 1
Staining of ES cells without or with expression of the antigens
GFP/Renilla luciferase with spleen cell supernatant.

| | | | | 129/SVEV + |
|---|---|---|---|---|
| | | | 129/SVEV + | Renilla |
| Strength of staining | H8L46 | 129/SVEV | GFP | luciferase |
| Negative | | 0, 1, 3, 4, 5 | 4 | 1, 4, 5 |
| Low | 1, 3, | | | 0, 3 |
| | 4* | | | |
| Low/modest | 0 | | | |
| Modest/strong | | | 1, 3 | |
| Strong/very strong | 2, 5 | 2 | 0, 2^{#}, 5 | 2 |

Preparation for staining and staining itself was performed as described above in material and Methods. Data reveal that only SCCS 2 has non-specific binding capacity and that in the other SCCS some level of antibody is present either directed to GFP or to Renilla luciferase and occasionally to both.
*indicates the SCCS of the mouse with the number noted.
^{#}Although staining cells positive there is no correlation with the level of GFP expression as is seen with the other SCCS staining positive.

Following the experiments utilizing stem cells we determined whether non-related cells such as STO (mouse embryonic fibroblasts, ATCC) cells and 293T (humane kidney, ATCC) cells are stained by the spleen cell supernatants. The 293T cells were further employed for transfection with either GFP or Renilla luciferase (non-secreted) expression constructs in order to obtain a higher expression level in a cellular background that was not used for immunization.

Both the STO and 293T cells were not recognized by the spleen cell supernatants of mouse-1, mouse-3 and mouse-4, while the spleen cells of mouse-0 and mouse-5 show a low staining of these cells (Table 2). Again spleen cell supernatant of mouse-2 recognizes the STO and 293T cells strongly underscoring the presence of non-specific antibodies (Table 2). Because this mouse was not immunized with the STO or 293T cells these a-specific antibodies were not induced by the immunization with ES cells and probably already present before the immunization. Upon transfection with GFP, recognition of 293T cells by the spleen cell supernatants of mouse-0, -1, -3, -4 was mild and depended on the strength of the GFP expression of the 293T cells. The spleen cell supernatant of mouse-2 and mouse-5 stained the cells strong to very strong, however only the supernatant of mouse-5 showed a correlation with the expression level of GFP.

The supernatant of mouse-2 stained every cell positive again indicating the presence of a-specific antibodies. 293T cells transfected with Renilla luciferase were not recognized by spleen cell supernatants-1 and -4, and low to modestly by spleen cell supernatant-0, -3 and -5. Again spleen cell supernant of mouse-2 showed a strong staining of all cells.

TABLE 2
Staining of STO/293T cells without or with expression of
the antigens GFP/Renilla luciferase with SCCS

| Strength of | | | | 293T + Renilla |
|---|---|---|---|---|
| staining | STO | 293T | 293T/GFP | luciferase |
| Negative | 1, 3, 4* | 1, 3, 4 | | 1, 4 |
| Low | 0, 5 | 0, 5 | | |
| Low/modest | | | 0, 1, 3, 4 | 0, 3, 5 |
| Modest/strong | 2 | 2 | | 2 |
| Strong/very | | | 2^{#}, 5 | |
| strong | | | | |

Preparation for staining and staining itself was performed as described above in material and Methods. Data reveal that only SCCS 2 has non-specific binding capacity and that in the other SCCS some level of antibody is present either directed to GFP or to Renilla luciferase and occasionally to both.
*indicates the SCCS of the mouse with the number noted.
^{#}Although staining cells positive there is no correlation with the level of GFP expression as is seen with the other SCCS staining positive.

Western Blot Analysis of the Polyclonal Antibody Sera from ES-GFP/sRenilla Immunized Mice.

To further analyze the specificity of the antibodies raised by immunization with GFP/sRenilla expressing H8L46-ES cells, we performed a western blot analysis with the Spleen cell supernatants of mice nrs-0 to -5. For this purpose a mixture of 50 ng recombinant Renilla-GST (Chemicon) and 25 ng recombinant GFP (Clontech) in 20 ul Laemmli sample buffer was subjected to 12% SDS-PAGE. After electrophoresis, the proteins were transferred by electro-blotting to PVDF transfer membrane (Millipore). Membranes were blocked in 5% non-fat milk in TBST (10 mM Tris-HCl pH8.0, 150 mM NaCl, 0.1% Tween-20) for 1 h at room temperature. Primary antibody incubation was performed overnight at 4° C. utilizing the commercially available GFP and Renilla luciferase antibodies (Chemicon, 1:1000), or the spleen cell culture medium (1:5) of the indicated mice. After incubation with the goat-anti-mouse-HRP secondary antibody (Jackson, 1:10.000) proteins were visualized by enhanced chemiluminescence utilizing ECL Western blotting detection reagent (Amersham).

The spleen cell supernatants of all 6 mice detected both the GFP and Renilla proteins on the blot thereby indicating the development of an antibody response against GFP and Renilla in all six mice. Mouse-0, mouse-3 and mouse-5 displayed the strongest signals against Renilla, whereas mouse-0 and mouse-5 also displayed strong signals against GFP. All six mice developed an immune response against GFP, although this protein is not actively secreted. Exposure of GFP to the immune system may however occur after cell death induced either by apoptotic processes during the embryonic development of the teratoma or by necrosis due to local unfavourable nutrient conditions in the teratoma.

Nevertheless, the results show that both secreted and intracellular expressed exogenous proteins are efficient in generating an immune response when expressed by a syngenic stem cell.

To further determine the specificity of the immune response, the Spleen Cell supernatants of mouse-0, mouse 3 and mouse-5 were also tested by Western blotting containing cell lysates of the background ES cell line (1), the transfected sRenilla/GFP ES line used for the immunization (2), the unrelated human embryonic kidney cell line 293T, un transfected (3) and transfected with GFP (4), cellular Renilla (5), or both cellular Renilla and GFP (6). The blotting procedure and the specificity of the antibodies were checked by a sample containing recombinant GFP and the recombinant Renilla-GST fusion protein (7). The results show clearly that all mice developed specific antibodies against Renilla and/or GFP. No significant response was observed against the syngenic ES background or the 293T cell lysate. However, the 293T cells transfected with the GFP and Renilla genes clearly stained proteins with the molecular weight sizes corresponding to Renilla and GFP thereby indicating the specificity of the immune response generated against Renilla and GFP induced by the immunization with ES GFP/sRenilla expressing syngenic ES cells.

### Conclusions

In these Examples we have analyzed the subcutaneous immunization with syngenic ES cells that were stably transfected and selected for highly expressing GFP and secreted Renilla Luciferase. The immunization and growth of the ES cells was followed by presence of secreted Renilla luciferase (sRenilla) in the serum of the mice. Three mice showed a very limited activity of sRenilla in the serum while their teratoma developed normal, indicating the formation of anti-sRenilla neutralizing antibodies in these mice. After euthanizing the mice, the presence and production of sRenilla and GFP specific antibodies was further investigated in the serum and in spleen cell culture supernatant.

Combining the serum samples with an external source of sRenilla revealed the presence of neutralizing sRenilla antibodies which was most prevalent in the three mice with the lowest sRenilla serum activity.

The serum samples of the mice were further tested on the presence of anti sRenilla and GFP antibodies by immune fluorescence on Renilla and GFP expressing cells. This experiment revealed again a strong correlation between anti-Renilla and anti-GFP immune fluorescent staining and the serum samples of the immunized mice, whereby only one mouse revealed a-specific antibodies against different cell-lines. To further determine the specificity of the immune response against GFP and sRenilla, the spleen culture antibody samples were subjected as a probe to western-blots containing complex protein samples. This experiment clearly revealed the presence of antibodies against the secreted Renilla in all six immunized mice. The immune response against the intracellular expressed GFP was less pronounced as to the sRenilla but clearly present in two mice. These results corresponded well to the immunefluorescent results obtained on transfected GFP/Renilla cells. Importantly, the western-blotting results also clearly showed that no immune response was raised against the syngenic ES cells. The only mouse showing non specific cross-reactivity to the ES cells also showed this to a non related cell line not used for the immunization indicating that this activity was probably present in this mouse before the immunization and was not induced by the immunization.

We therefore conclude that the immunization of mice with syngenic ES cells expressing non-self proteins is an efficient and reliable way to induce and immune response against these non-self proteins. Methods according to the invention can be applied for either extracellular proteins or intracellular expressed proteins. Methods according to the invention make it possible to raise an immune response against a complex epitope composed of multiple proteins.

Methods according to the invention may also be used to raise an immune response against multiple proteins at once, in order to obtain a broad protection against, for instance, a pathogen and/or a malignant cell.

### Example 3

The Isolation of Mouse Monoclonal Antibodies Specific for GFP and Renilla using the Stem Cell Immunization Method.

In Example 1 we have shown that the stem cell immunization method can be used to induce a strong antibody response against non-self antigens.

Now we have used the stem cell immunization method for the generation of monoclonal antibodies specific for either GFP or Renilla. For this means, mice of the strains OLA129 and Balb-c were mated and the F1-offspring was used at an age of two months for immunization with the H8L46 embryonic stem (ES) cell line expressing secreted Renilla luciferase and non-secreted GFP.

The monoclonal antibodies were generated using a standard technique in which antibody producing hybridoma cells are generated by the fusion of immortal Sp2(0) myeloma cells with the B-cells of an immunized mouse (materials & methods).

### Material & Methods

### Materials

DMEM high glucose+Pyruvate (Gibco, 31966). Penicillin/streptomycin-100× (Gibco, 15140). FBS (Gibco 10082). PEG (Sigma, P7777). HAT (Sigma, H0262-10VL). HT (Sigma, H0137-10VL). Hybridoma Cloning Factor (PAA, 501-015, HCF). 8-azaguanine (Sigma A5284_{—}10VL). Culture medium myeloma cells: DMEM+10% FBS+Penicilinne/Streptamycine (1×)+glutamax(1×)+130 μM 8-azaguanine. Sp2(0) Myeloma cells.

### Antibodies and Proteins.

The antibodies used to validate the ELISA were anti-Renilla monoclonal antibody MAB4410 (Millipore), anti-GFP monoclonal antibody MAB3580 (Millipore). The conjugate used to detect the monoclonal antibodies was the peroxidase-conjugated Affinipure Goat anti-mouse IgG+IgM (H+L) and obtained from Jackson-Laboratories (cat.nr. 115-035-068). The recombinant Renilla luciferase was obtained from chemicon (novalite, cat.nr. 4400. The recombinant GFP protein was purchased from Polysciences (catnr. 24240).

### ELISA

The ELISA used to screen the supernatants of the hybridoma cultures was performed by standard procedures. Briefly, ELISA plates(Greiner) were coated overnight at 4° C. with recombinant GFP or Renilla protein at a concentration of 50 ng/ml coating buffer (0.1 M Carbonate/bicarbonate buffer, pH=9.6). Next day, the plates were blocked for one hour using 150 μl per well blocking buffer (1% casein in coating buffer). After blocking the plates were washed with PBS-Tween (0.05%) and the wells are filled with 100 μl of hybridoma culture medium and incubated overnight at 4° C. Next day, the plates were washed three times with PBS-Tween(0.05%) and the wells were filled with 100 μl Goat anti-Mouse conjugate solution per well (1:10.000 diluted in blocking buffer) and incubated for 2 hours at room temperature. Next the wells were washed with PBS-Tween (0.05%) and incubated with 100 μl substrate per well (0.1% 100 TMB (Sigma catnr. T8768) in Na-Acetate buffer (pH=5.5)+2.5 μl 30% H202. The colour development was stopped by the addition of 100 μl 0.8 M H2SO4. The absorbance of the colour reaction was measured at 450 nm using an ELISA reader.

### Culture Media

General SP2/0 culture medium: DMEM with glutamax including 50 ml FBS, 5 ml of 100× Penicillin/Streptomycin.

Serum free medium: DMEM+5 ml Penicillin/Streptomycin (100×)

HAT medium(50×): 1 vial HAT was dissolved in 10 ml serum free medium. Before use 2 ml of this stock solution was added to 98 ml of complete medium containing 10% FCS. Final working concentration: 100 μM hypoxanthine, 0.4 μM aminopterin, 16 μM thymidine.

HT medium (50×): 1 vial HT was dissolved in 10 ml serum free medium. Before use 2 ml of this stock solution was added to 98 ml of complete medium containing 10% FCS. Final working concentration: 100 μM hypoxanthine, 16 μM thymidine.

PEG (50%): The PEG1300-1600 was melted in a water bath higher at a temperature of 50° C. for more than 5 minutes and subsequently autoclaved for 5 minutes to melt completely. Five ml of serum free medium was added to the melted PEG to obtain a 50% solution.

Final culture medium: 500 ml DMEM+5 ml P/S (100×)+50 ml FBS+10% HCF.

### Hybridoma Procedure

The Sp2(0) myeloma cells are collected from an exponential growing culture. The immunized mouse is sacrificed and the spleen is dissected and subsequently minced through a fine mash in order to isolate the spleen cells. Both the Sp2(0) and the spleen cell preparation are collected in serum free medium and mixed at a ratio of 2-5 spleen cells to 1 Sp2(0) cell. To fuse the splenocytes and Sp2(0) cells remove the medium and add 0.5 ml PEG over a period of 30 seconds. Next, the cells are gradually mixed with 22 ml of serum free medium over a period of 8 minutes. The fused cells are left on ice for 5 minutes and are subsequently centrifuged. The supernatant is removed and the cells are supplemented in 150 ml HAT-medium. The cells are dispersed over ten 96 wells plates and wrapped in foil to prevent evaporation. After 1 week the HAT-medium is gradually replaced by HT-medium by the addition of 50 μl of HT-medium to each well, every 3-4 days. After three weeks the hybridoma containing wells can be screened for antibody production.

### Immunization

At day-1, the Ola129/Balb-c (F1) mice were subcutaneously immunized with 2×10⁶ H8L46 ES cells. After 5 weeks the mice received an intraperitoneal injection of 2.5×10⁶ H8L46 cells in order to boost the immune system.

### Results

One mouse with a teratoma was selected for the hybridoma procedure as outlined in materials and methods. Briefly, the spleen was dissected and used for the isolation of spleen cells. The splenocytes were fused with the Sp2(0) cell line and the cell mixture was supplemented in HAT-medium, divided over ten 96 wells culture dishes and cultured at 37° C. and 5% CO2. After 1 week the HAT-medium was gradually replaced by HT-medium.

After three weeks 250 wells showed growth of well proliferating hybridomas which were tested for the production of antibodies against secreted Renilla and GFP.

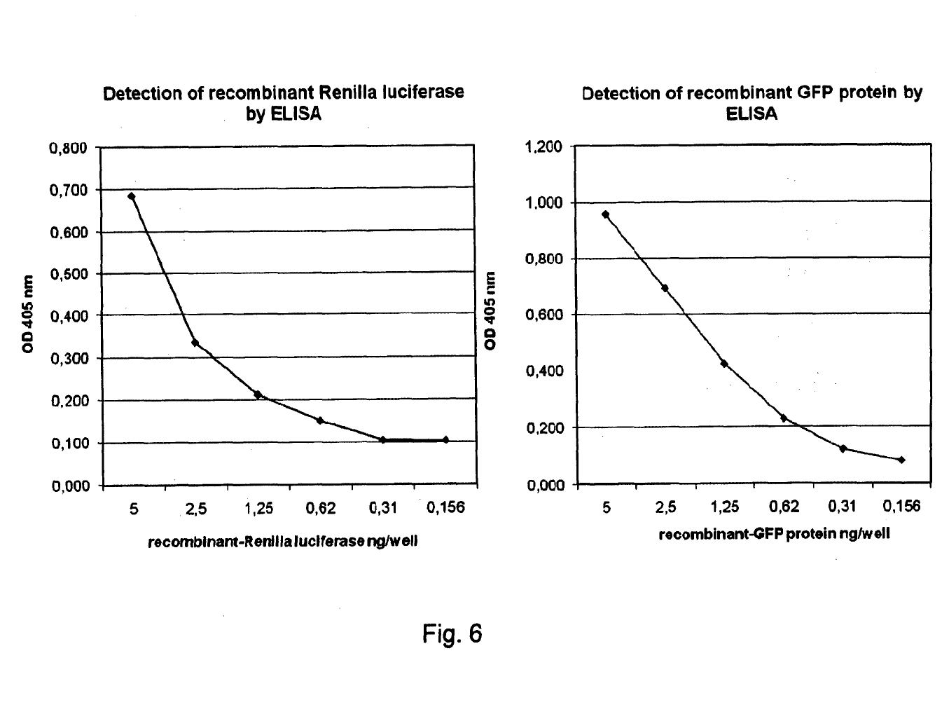

The hybridoma supernatants were screened by ELISA for antigen specific antibodies against GFP and Renilla. The ELISA was previously validated using recombinant Renilla and GFP and commercial antibodies against Renilla and GFP (FIG. 6).

After several rounds of independent screening of the supernatants by Elisa three hybridomas were found to produce monoclonal antibodies against Renilla and 6 hybridomas produced monoclonal antibodies against GFP (Table 3).

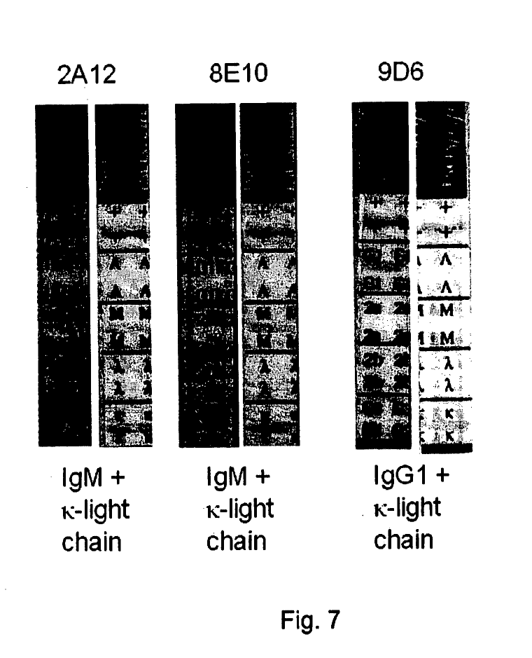

Three monoclonals with affinity for GFP were used to determine the isotype of the Heavy and light chains. Two of the monoclonals appeared to belong to the IgM class whereas one monoclonal belonged to the IgG1 class (FIG. 7).

### Conclusions

In this experiment we have demonstrated that the stem cell immunization method is valuable for the generation of monoclonal antibodies with the hybridoma technique.

In this experiment we have isolated nine independent hybridomas of which three were specific for Renilla luciferase and of which six produced monoclonal antibodies specific for GFP. Two of the antiGFP monoclonal antibodies had an IgM isotype, whereas one had an IgG1 isotype. We conclude that the stem cell immunization method is very useful for the induction of a specific immune response and the generation of monoclonal antibodies using hybridoma technology.

TABLE 3
Specificity of the antibodies produced by the hybridomas
obtained with the stem cell immunization method.

| | | Heavy | Light |
|---|---|---|---|
| Clone ID | Affinity | chain | chain |
| 2A12 | Anti-GFP | IgM | κ |
| 8E10 | Anti-GFP | IgM | κ |
| 9D6 | Anti-GFP | IgG1 | κ |
| 2A3 | Anti-GFP | N.D. | N.D. |
| 4G5 | Anti-GFP | N.D. | N.D. |
| 9F3 | Anti-GFP | N.D. | N.D. |
| 3G5 | Anti-Renilla | N.D. | N.D. |
| 5A4 | Anti-Renilla | N.D. | N.D. |
| 10E7 | Anti-Renilla | N.D. | N.D. |

N.D.: Not Determined

### Example 4

Here we have applied a new vaccination technique in which we use embryonic stem cells (ES cells) as a vehicle to express an antigen. Embryonic stem cells are undifferentiated cells with a normal karyotype that have the ability to differentiate to all possible cell-types. When differentiation of these ES cells is induced and subsequently these cells are injected into a mouse strain from which the ES cells originally have been isolated, i.e. an isogenic strain, the ES cells form a small tissue that is considered as “normal or self” to the host. However, the ES cells express one or multiple pathogenic proteins which activate the immune system. This way of vaccination has multiple advantages such as in vivo expression of complex and difficult to isolate antigens, processing of the antigen by the eukaryotic cell machinery with accompanying post-translation modifications, long persistence of the immunogen and the possibility to induce a broad immune response including the activation of cytotoxic T cells, T helper cells and antibody production.

In order to demonstrate that embryonic stem cells can be used as a vehicle to express exogenous proteins that induce an immune response, we have transfected mouse embryonic stem cells with DNA constructs expressing the human form of gp100, a melanoma antigen which is associated with malignant melanoma's in humans. Since the used ES cells are isogenic for the used mouse strain (C57BL/6 in this example), human gp100 is the only protein that is non-self for these mice. After subcutaneous and/or intraperitoneal administration of the ES cells expressing human gp100, the protein is secreted and thus fully exposed to the immune system. Vaccinated mice were subsequently infected with the mouse melanoma cell line B16 subline F10 that are transfected with human gp100 and stably express this protein.

We found that the growth of the melanomas in vaccinated mice was considerably reduced as compared to control mice that were vaccinated with empty ES cells.

These results show that vaccination with ES cells expressing pathogenic antigens is highly effective in inducing an immune response and induce (at least partial) protection.

### Material and Methods:

Generation of Embryonic Stem Cells Constitutively Expressing Human gp100.

ES cells from the C57BL/6 mouse strain were transfected with an expression construct encoding full length gp100 fused to renilla luciferace under control of the strong chimeric beta-actin/CMV (CAG) promoter. The selection of stably transfected ES cells was performed with Hygromycin. The stably transfected gp100 ES clones were screened for high expression of the gp100-Renilla fusion protein by monitoring the luminescence activity using a luminometer (berthold) and the stop&glow assay (promega). The ES clone showing the strongest luminescence signal was used for vaccination.

Culture of the ES Cells Expressing gp100 and Preparation of Inoculum

Mice were vaccinated with ES cells in an inoculum that was prepared as follows: stable ES cells expressing the human gp100-renilla fusion protein were thawed and cultured in 162 cm² culture flask (Greiner) that were coated with 0.1% gelatin (Sigma) in complete culture medium (DMEM culture medium (Gibco) containing per 500 ml flask 2 mM glutamine, 1 unit Penicillin, 100 μg Streptomycin, 5 ml of a 100× solution Non-Essential Amino Acids, 5 ml of a 100× solution of Nucleosides, 5 ml of a 100× solution of Beta 2-Mercaptoethanol (all supplied by Millipore), 10⁵ units LIF (Chemicon), and 50 ml FCSi (Sigma)). After 1 day, the culture medium was replaced by complete culture medium without LIF. The absence of LIF induced differentiation of the ES cells. After another two days of culturing at 37° C. in a CO2 incubator, the ES cells were collected by trypsin/EDTA (Gibco) treatment. The cells were washed 2 times with phosphate buffered saline (PBS, Gibco) and a cell suspension of 5-10×10⁶ ES cells per 200 μl in PBS was prepared.

### Vaccination of the Mice

Animals were housed under the guidelines of the Animal facility of the Leiden University Medical Centre (PDC-LUMC) according to the Dutch law. C57BL/6 mice were commercially obtained from Charles River (Sulzfeld, Germany).

To vaccinate the mice, they were challenged a first time by subcutaneously injection of in total 8×10⁶ ES cells in 200 μl divided over 4 sides near the left or right hind and forelegs. Four weeks later, mice were boosted a second time by intraperitoneal injection of 5×10⁶ ES cells in 200 μl PBS. A third and last vaccination was performed 7 weeks from the start by injecting in total 10×10⁶ ES cells subcutaneously at two sides in the flanks. Animals were examined weekly and ones weekly blood was drawn by tail incision and collected in EDTA coated vials (Sarstedt).

### Melanoma Infection of the Mice.

The human gp100 gene was cloned in a pUC-19 based expression vector under control of the hybrid chicken beta-actin/CMV promoter and a SV-40 polyadenylation signal sequence, and was subsequently used to transfect the murine melanoma cell line B16 subline F10 together with a vector carrying the hygromycin selectable gene in order to screen for stable hygromycin resistant clones. Among the stably transfected clones the highest expressing gp100 clones was selected by semi-quantitative RT-PCR and this clones was expanded and used for the inoculation of the mice. Five days after the last vaccination, all mice were challenged subcutaneously with 1×10⁵ live B16F10 melanoma cells expressing the human gp100. Mice were sacrificed two weeks later and melanomas were isolated. The size of growing melanomas was determined by a weighting the tumors.

### Results

### Body Weight of Vaccinated Mice

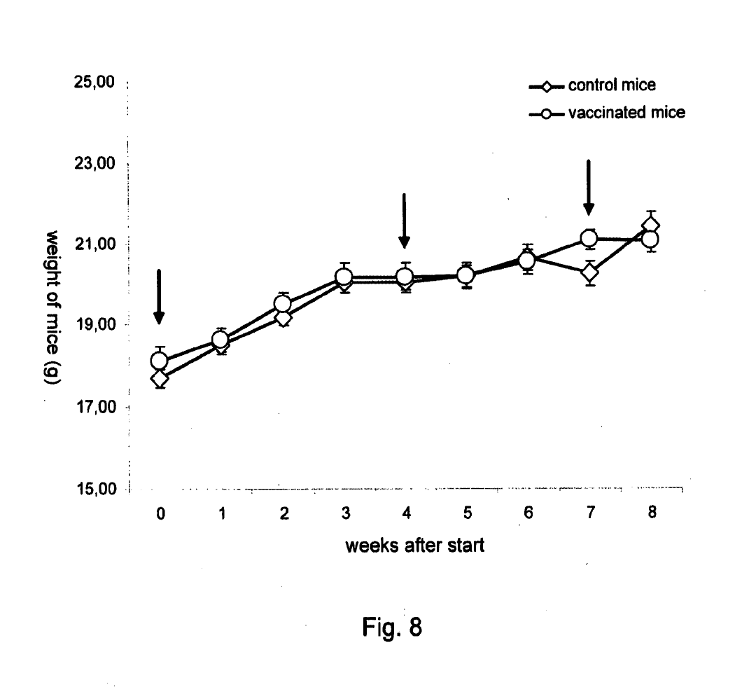

Female mice of 12 weeks of age were vaccinated in total three times with 5-10×10⁶ ES-cells expressing gp100. Weekly observation of the mice did not reveal any negative effects of the vaccination procedure. This was also reflected in the bodyweight of the mice. All mouse showed a normal body weight curve that was not influenced in the control group or in the treatment group by the vaccination (FIG. 8).

Vaccination with ES Cells Expressing Human gp100 Reduces the Growth of B16F10 Melanocytes

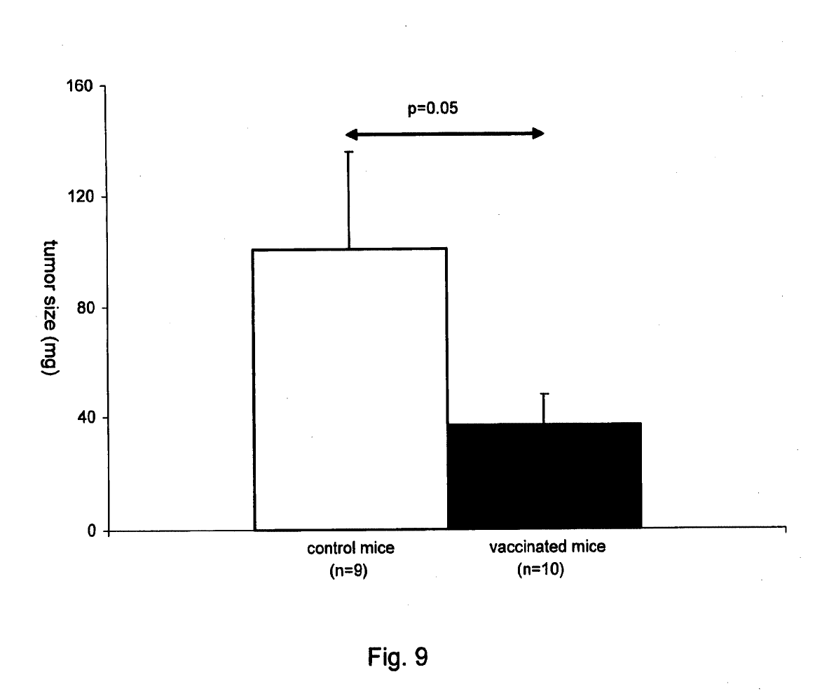

To study the efficacy of vaccination with isogenic ES cells expressing human gp100, mice were vaccinated and subsequently challenged with B16F10 melanocytes also expressing the human gp100. As is shown in Table 4 and FIG. 9, the control group of mice all developed a tumor within two weeks varying in size from 6 mg to as large as 347 mg with a mean weight of 100 mg. One mouse in the control group even developed metastases of the melanoma (data not shown). In contrast, the vaccinated mice showed a strong reduction in tumor growth. The tumor size in these mice varied from no tumor formation at all (one mouse) to a maximal size of 114 mg. The mean weight of the tumors was reduced by 63% to a mean weight of 37 mg as compared to the control group. Statistical analysis according to the Mann-Whitney U test, reveals that this reduction in growth of the tumors reaches significant levels (p=0.05) indicating strong efficacy of this immunization method.

### CONCLUSIONS

In this study we have examined the efficacy of vaccination with ES cells that were stably transfected with the immunogenic protein gp100. We have found that the vaccination with ES cells expressing gp100 is effective in priming and activating the immune system and in counteracting the growth of aggressive growing murine melanocytes expressing human gp100.

Prior to vaccination, the cultured ES cells were induced to differentiate in order to prevent subcutaneous teratoma formation. In the vaccinated mice and in the control group no teratoma formation was observed in any of the mice. Despite the absence of teratomas the vaccine showed strong efficacy against the melanoma tumor formation, demonstrating that teratoma formation is not an absolute requirement for inducing an effective immune response. In addition, the used ES cells in this example originated from the C57BL/6 mouse strain and thus originate from a different strain and genetic background than the ES cells used in Examples 1 and 3. The vaccination method according to the present invention is therefore applicable with a multitude of ES cells obtained from different genetic backgrounds. This study shows that the stem cell immunization method is useful as a new vaccination technique which is widely applicable for counteracting many different pathogens.

TABLE 4
Tumor size in control mice and ES cell vaccinated mice

| | |
|---|---|
| control mice | vaccinated mice |

| | tumor size | | tumor size |
|---|---|---|---|
| mouse # | (mg) | mouse # | (mg) |
| 1 | 100 | 1 | 28 |
| 2 | 54 | 2 | 0 |
| 3 | 66 | 3 | 27 |
| 4 | 186 | 4 | 54 |
| 5 | 347 | 5 | 37 |
| 6 | 51 | 6 | 39 |
| 7 | 31 | 7 | 114 |
| 8 | 62 | 8 | 7 |
| 9 | 6 | 9 | 60 |
| | | 10 | 4 |

1. A method for inducing and/or enhancing an immune response in an animal against an antigen of interest, comprising exposing said animal to a syngenic stem cell, a syngenic progenitor cell and/or a syngenic dedifferentiated cell, which cell comprises a nucleic acid sequence encoding said antigen of interest, wherein said antigen of interest is exogenous to said animal and/or wherein said antigen of interest is not encoded by the germ line of said animal.

2. A method according to claim 1, wherein said cell is capable of forming a teratoma in said animal.

3. A method according to claim 1, wherein said cell is mitotically inactivated.

4. A method according to claim 1, wherein said cell is killed.

5. A method according to claim 1, wherein said cell comprises an embryonic stem cell, a primordial germ cell, a multipotent adult progenitor cell (MAPC), an embryonic progenitor cell, a mesenchymal stem cell, a mesenchymal progenitor cell, a haematopoietic stem cell, a hematopoietic progenitor cell, a lung stem cell, a lung progenitor cell, a dedifferentiated somatic cell and/or an immortalized somatic cell.

6. A method according to claim 1, wherein said antigen of interest comprises an antigen of a pathogenic organism.

7. A method according to claim 1, wherein said antigen of interest comprises an antigen expressed by a malignant cell.

8. A method according to claim 1, wherein said antigen of interest cannot be purified without disturbing its structure.

9. A method according to claim 1, wherein said animal comprises a mammal or a bird, preferably a primate, an ape, a rodent, a mouse, a rat, a rabbit, a llama, a donkey, a goat, a pig, a cow, a horse, a chicken or a pet.

10. A method according to claim 1, wherein said animal comprises a human individual.

11. A method according to claim 1, further comprising obtaining from said animal an antibody, a polyclonal antibody, a B-lymphocyte, or a T-lymphocyte specifically directed against said antigen of interest.

12. A method according to claim 11, further comprising immortalizing said obtained B-lymphocyte or T-lymphocyte.

13. A method according to claim 12, wherein said B-lymphocyte is used to generate monoclonal antibodies.

14. A method according to claim 11, further comprising obtaining antibodies produced by said B-lymphocyte or produced by a cell comprising a gene encoding an Ig heavy chain and/or an Ig light chain of said B-lymphocyte.

15. A method according to claim 11, further comprising preparing a medicament or prophylactic agent comprising said B-lymphocyte, T-lymphocyte and/or said antibody, or a functional part or derivative thereof.

16. A method according to claim 11, further comprising administering said antibody, B-lymphocyte, T-lymphocyte, functional part, derivative, medicament or prophylactic agent to an individual suffering from, or at risk of suffering from, a disorder associated with the presence of said antigen of interest.

17. An antibody, B-lymphocyte, T-lymphocyte, medicament or prophylactic agent obtained with a method according to claim 11.

18. An antibody or a T-lymphocyte obtained by a method according to claim 11, or a functional part or derivative thereof, for use as a medicament or prophylactic agent.

19. (canceled)

20. A stem cell or progenitor cell or dedifferentiated cell comprising a nucleic acid sequence encoding an antigen of interest, for use as a medicament or prophylactic agent, wherein said stem cell or progenitor cell or dedifferentiated cell is syngenic to the recipient and wherein said antigen of interest is exogenous to the recipient and/or wherein said antigen of interest is not encoded by the germ line of the recipient.

21.-23. (canceled)

24. A medicament or prophylactic agent comprising a stem cell or a progenitor cell or a dedifferentiated cell and a suitable carrier, diluent or excipient, wherein said stem cell or progenitor cell or dedifferentiated cell is syngenic to the recipient and wherein said stem cell or progenitor cell or dedifferentiated cell comprises a nucleic acid sequence encoding an antigen of interest which is exogenous to the recipient or which is not encoded by the germ line of the recipient.

25. A non-human animal wherein an immune response is raised against an exogenous antigen of interest, or against an antigen of interest which is not encoded by the germ line of said animal, said non-human animal comprising a stem cell or progenitor cell or dedifferentiated cell that is syngenic to said animal, said cell comprising a nucleic acid sequence encoding said antigen of interest.

26. A method for counteracting and/or preventing a disorder associated with the presence of an antigen of interest, comprising administering to an animal suffering from, or at risk of suffering from, said disorder a therapeutically effective amount of a syngenic stem cell or syngenic progenitor cell or syngenic dedifferentiated cell comprising said antigen of interest, or a therapeutically effective amount of an antibody or T-lymphocyte specifically directed against said antigen of interest obtainable by a method according to claim 11, or a functional part or derivative of said antibody or T-lymphocyte.

27. A method for vaccinating an animal against an antigen of interest or multiple antigens of interest, comprising administering to said animal a syngenic stem cell or syngenic progenitor cell or syngenic dedifferentiated cell comprising at least one nucleic acid sequence encoding said antigen of interest or encoding multiple antigens of interest.
